(19) United States
(12) Patent Application Publication
(10) Pub. No.: US 2006/0121113 A1
(43) Pub. Date: Jun. 8, 2006

(54) Pharmaceutical composition containing 6-dimethylaminomethyl-1-(3-methoxyphenyl)-cyclohexane-1,3-diol with delayed active ingredient release

(75) Inventor: Johannes Bartholomaeus, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/334,344

(22) Filed: Jan. 19, 2006

Related U.S. Application Data
(63) Continuation of application No. PCT/EP04/08081, filed on Jul. 20, 2004.

(30) Foreign Application Priority Data
Jul. 24, 2003 (DE) 103 33 835.7

(51) Int. Cl. A61K 31/137; A61K 9/22

(52) U.S. Cl. 424/468; 514/649

(57) ABSTRACT

Pharmaceutical compositions with delayed release which contains 6-dimethylaminomethyl-1-(3-methoxyphenyl)-cyclohexane-1,3-diol or a pharmaceutically acceptable salt thereof in a matrix with delayed active ingredient release, wherein the matrix contains 1 to 80 wt. % of one or more hydrophilic or hydrophobic polymers as pharmaceutically acceptable matrix formers and exhibits the following in vitro release rate: 3-35 wt. % (relative to 100 wt. % of active ingredient) of 6-dimethylaminomethyl-1-(3-methoxyphenyl)-cyclohexane-1,3-diol released after 0.5 hours, 5-50 wt. % after 1 hour, 10-75 wt. % after 2 hours, 15-82 wt. % after 3 hours, 30-97 wt. % after 6 hours, more than 50 wt. % after 12 hours, more than 70 wt. % after 18 hours, and more than 80 wt. % after 24 hours.

### CROSS REFERENCE TO RELATED APPLICATIONS

This application is a continuation of international patent application no. PCT/EP2004/008081, filed Jul. 20, 2004, designating the United States of America, and published in German as WO 2005/009329 on Feb. 3, 2005, the entire disclosure of which is incorporated herein by reference. Priority is claimed based on Federal Republic of Germany patent application no. DE 103 33 835.7, filed Jul. 24, 2003.

### FIELD OF THE INVENTION

The invention relates to a pharmaceutical composition with delayed active ingredient release which contains 6-dimethylaminomethyl-1-(3-methoxyphenyl)-cyclohexane-1,3-diol or a pharmaceutically acceptable salt thereof in a matrix.

### BACKGROUND OF THE INVENTION

6-Dimethylaminomethyl-1-(3-methoxyphenyl)-cyclohexane-1,3-diol is known from EP 0 753 506 B1 or U.S. Pat. No. 5,733,936 as an analgesically active pharmaceutical preparation and can be administered orally. Conventional formulations for oral administration of 6-dimethylaminomethyl-1-(3-methoxyphenyl)-cyclohexane-1,3-diol result in a relatively rapid release of the active ingredient in the gastrointestinal tract, such that it is also rapidly analgesically active. However, its action can also be observed to subside relatively rapidly. Treatment of severe, chronic pain with 6-dimethylaminomethyl-1-(3-methoxyphenyl)-cyclohexane-1,3-diol has accordingly hitherto entailed administering the pharmaceutical preparation at relatively short intervals, for example three to four times daily, in order to ensure a sufficient active ingredient concentration in the patient's blood plasma. However, the necessity of administering frequent doses often results in errors in taking and in undesirable fluctuations in plasma concentration, which have a negative impact on patient compliance and therapeutic benefit, in particular in the treatment of chronic pain. A pharmaceutical dosage form with delayed release (controlled release formulation) for oral administration of the active ingredient 6-dimethylaminomethyl-1-(3-methoxyphenyl)-cyclohexane-1,3-diol is thus desirable.

Controlled release formulations for a large number of different active ingredients are generally known in the prior art. Controlled release is conventionally achieved inter alia by coated formulations and matrix formulations.

In the case of coated controlled release formulations, as are described for example in DE 36 25 458 A1, the core of a pharmaceutical composition containing an active ingredient is provided with a coating of one or more hydrophilic and/or hydrophobic polymers which delay the release of the active ingredient.

In matrix controlled release formulations, the active ingredient is held in a matrix formed from one or more matrix materials which controls the release of the active ingredient. DE 33 09 516 A1, for example, discloses a process for the production of matrix formulations with hydroxypropylmethylcellulose (HPMC) as matrix material and with partially delayed release of the active ingredient, wherein the matrix material constitutes no more than one third of the weight of the formulation and consists of at least one hydroxypropylmethylcellulose, which has a methoxy content of 16-24 wt. %, a hydroxypropyl content of 4-32 wt. % and a number average molecular weight of at least 50,000. The formulations disclosed in DE 33 09 516 A1 contain HPMCs with viscosities (in 2 wt. % aqueous solution at 20° C.) of between 15 and 30,000 cPs (15 to 30,000 mPa·s). DE 33 09 516 A1 does not disclose release behavior which is independent of the pH value of the dissolution medium.

### DESCRIPTION OF THE INVENTION

It is accordingly an object of the present invention to provide a pharmaceutical formulation containing 6-dimethylaminomethyl-1-(3-methoxyphenyl)-cyclohexane-1,3-diol with delayed active ingredient release.

Said object is achieved by a pharmaceutical formulation with delayed release which contains 6-dimethylaminomethyl-1-(3-methoxyphenyl)-cyclohexane-1,3-diol or a pharmaceutically acceptable salt thereof in a matrix with delayed active ingredient release, wherein the matrix contains 1 to 80 wt. %, preferably 5 to 80 wt. % of one or more hydrophilic or hydrophobic polymers as pharmaceutically acceptable matrix formers, or matrix forming polymers, and exhibits the following in vitro release speed, measured using the European Pharmacopoeia (“Ph. Eur.”) paddle method at 75 rpm in a buffer (in compliance with Ph. Eur.) at a pH value of 6.8 at 37° C. and with detection by UV spectrometry:
3-35 wt. % of 6-dimethylaminomethyl-1-(3-methoxyphenyl)-cyclohexane-1,3-diol (relative to 100 wt. % of active ingredient) released after 0.5 hours,
5-50 wt. % of 6-dimethylaminomethyl-1-(3-methoxyphenyl)-cyclohexane-1,3-diol released after 1 hour,
10-75 wt. % of 6-dimethylaminomethyl-1-(3-methoxyphenyl)-cyclohexane-1,3-diol released after 2 hours,
15-82 wt. % of 6-dimethylaminomethyl-1-(3-methoxyphenyl)-cyclohexane-1,3-diol released after 3 hours,
30-97 wt. % of 6-dimethylaminomethyl-1-(3-methoxyphenyl)-cyclohexane-1,3-diol released after 6 hours,
more than 50 wt. % of 6-dimethylaminomethyl-1-(3-methoxyphenyl)-cyclohexane-1,3-diol released after 12 hours, ore than 70 wt. % of 6-dimethylaminomethyl-1-(3-methoxyphenyl)-cyclohexane-1,3-diol released after 18 hours, and
more than 80 wt. % of 6-dimethylaminomethyl-1-(3-methoxyphenyl)-cyclohexane-1,3-diol released after 24 hours.

The present invention also provides a pharmaceutical formulation with delayed release, which contains 6-dimethylaminomethyl-1-(3-methoxyphenyl)-cyclohexane-1,3-diol or a pharmaceutically acceptable salt thereof in a matrix with delayed active ingredient release, wherein the matrix contains 1 to 80 wt. % of one or more hydrophilic or hydrophobic polymers as pharmaceutically acceptable matrix formers and exhibits the following in vitro release speed, measured using the Ph. Eur. paddle method at 75 rpm in a buffer (according to Ph. Eur.) at a pH value of 6.8 at 37° C. and with detection by UV spectrometry:
3-60 wt. % (relative to 100 wt. % of active ingredient) of 6-dimethylaminomethyl-1-(3-methoxyphenyl)-cyclohexane-1,3-diol released after 0.5 hours,
5-70 wt. % of 6-dimethylaminomethyl-1-(3-methoxyphenyl)-cyclohexane-1,3-diol released after 1 hour,
10-75 wt. % of 6-dimethylaminomethyl-1-(3-methoxyphenyl)-cyclohexane-1,3-diol released after 2 hours,
15-82 wt. % of 6-dimethylaminomethyl-1-(3-methoxyphenyl)-cyclohexane-1,3-diol released after 3 hours,
30-97 wt. % of 6-dimethylaminomethyl-1-(3-methoxyphenyl)-cyclohexane-1,3-diol released after 6 hours,
more than 50 wt. % of 6-dimethylaminomethyl-1-(3-methoxyphenyl)-cyclohexane-1,3-diol released after 12 hours,
more than 70 wt. % of 6-dimethylaminomethyl-1-(3-methoxyphenyl)-cyclohexane-1,3-diol released after 18 hours, and
more than 80 wt. % of 6-dimethylaminomethyl-1-(3-methoxyphenyl)-cyclohexane-1,3-diol released after 24 hours.

It has surprisingly been found that the formulations according to the invention release the active ingredient 6-dimethylaminomethyl-1-(3-methoxyphenyl)-cyclohexane-1,3-diol in delayed manner when orally administered and are accordingly suitable for administration at intervals of at least 12 hours optionally also at intervals of at least 24 hours. The formulation according to the invention accordingly permits pain therapy which requires the administration of the analgesic 6-dimethylaminomethyl-1-(3-methoxyphenyl)-cyclohexane-1,3-diol only once daily, for example at intervals of 24 h, or twice daily, preferably at intervals of 12 hours, in order to ensure an adequate plasma concentration of the active ingredient. Such a duration of action and the maintenance of an adequate blood plasma level is demonstrated by simulation studies and experimental investigations.

It is particularly surprising in this connection that the formulation according to the invention ensures extended therapeutic effectiveness over a relatively long period (at least 12 to 18, optionally 24 hours) not only due to the delayed release, but at the same time also due to the resultant favorable utilization of the long half-life of the active metabolites which are formed. Thanks to this half-life, release need only extend over 12 to 18 hours, in order to achieve adequate effectiveness in pain treatment over 24 hours. This formulation is thus surprisingly particularly suitable to be taken once daily, something which is distinctly more difficult to achieve with other comparable formulations. By taking the analgesic in the formulation according to the invention, the patient suffering from pain can thus effectively provide acute pain relief and simultaneously, without any further action, provide effective treatment over a longer period simply by taking the formulation regularly at intervals of 24 (or also 12) hours.

The active ingredient of the formulation according to the invention is contained in a matrix with delayed release. It is, however, also conceivable for the active ingredient to be contained in a matrix with conventional release behavior and for delayed release to be achieved by means of a controlled release coating.

Another possibility is for delayed release behavior to be achieved by an osmotically driven release system.

In the event that the composition according to the invention contains a matrix with delayed release, the matrix comprises 1-80 wt. % of one or more hydrophilic or hydrophobic polymers as pharmaceutically acceptable matrix formers, for example gums, cellulose ethers, cellulose esters, acrylic resins, protein-derived materials, fats, waxes, fatty alcohols or fatty acid esters. When hydrophilic polymers are used as the matrix former, it is preferred for the matrix to comprise 5 to 80 wt. % of matrix former.

The present invention also provides a pharmaceutical formulation which contains 6-dimethylaminomethyl-1-(3-methoxyphenyl)-cyclohexane-1,3-diol or a pharmaceutically acceptable salt thereof in a matrix with delayed active ingredient release, wherein the matrix contains 1 to 80 wt. %, in particular 5 to 80 wt. % of one or more hydrophilic or hydrophobic polymers as pharmaceutically acceptable matrix formers and which is characterized in that it comprises as pharmaceutically acceptable matrix cellulose ethers and/or cellulose esters which exhibit a viscosity in a 2 wt. % aqueous solution at 20° C. of 3,000 to 150,000 mPa·s. (Viscosity is here determined by means of capillary viscosimetry in accordance with Pharm. Eu.). The compositions exhibit the above-stated release profile according to the invention.

Preferably used pharmaceutically acceptable matrix formers are cellulose ethers and/or cellulose esters which have a viscosity in a 2 wt. % aqueous solution at 20° C. of between 10,000 and 150,000 mPa·s, in particular between 50,000 mPa·s and 150,000 mPa·s.

Particularly suitable pharmaceutically acceptable matrix formers are selected from the group of hydroxypropylmethylcelluloses (HPMC), hydroxyethylcelluloses, hydroxypropylcelluloses (HPC), methylcelluloses, ethylcelluloses and carboxymethylcelluloses, and are in particular selected from the group of HPMCs, hydroxyethylcelluloses and HPCs. HPMCs with a viscosity of approximately 100,000 mPa·s, measured in a 2 wt. % aqueous solution at 20° C., are most highly preferred.

The active ingredient 6-dimethylaminomethyl-1-(3-methoxyphenyl)-cyclohexane-1,3-diol may be present not only as such, i.e. as the free base, but also in the form of a pharmaceutically acceptable salt, for example as hydrochloride. Production of the free base is known from EP 0 753 506 A1 or U.S. Pat. No. 5,733,936. Insofar as EP 0 753 506 A1 or U.S. Pat. No. 5,733,936 does not also disclose the production of pharmaceutically acceptable salts, such as of the hydrochloride, such salts are obtainable from the free base by means of methods generally known in the prior art.

6-Dimethylaminomethyl-1-(3-methoxyphenyl)-cyclohexane-1,3-diol has centers of asymmetry, such that the compound may assume the form of different stereoisomers. In the formulation according to the invention, 6-dimethylaminomethyl-1-(3-methoxyphenyl)-cyclohexane-1,3-diol may be present not only as a mixture of all stereoisomers in any desired mixing ratio, but also as a mixture of two or three or more stereoisomers in any desired mixing ratio or in stereoisomerically pure form. Stereoisomers are here in particular taken to mean enantiomers or diastereomers. The racemic mixture (1RS,3RS,6RS)-6-dimethylaminomethyl-1-(3-methoxyphenyl)-cyclohexane-1,3-diol is preferred in the formulation according to the invention. For the purposes of the present invention, the “active ingredient” or possibly usable forms of 6-dimethylaminomethyl-1-(3-methoxyphenyl)-cyclohexane-1,3-diol should accordingly be taken to comprise 6-dimethylaminomethyl-1-(3-methoxyphenyl)-cyclohexane-1,3-diol as a racemic mixture or as a mixture of various of the stereoisomers thereof in any desired mixing ratio or as one of the pure stereoisomers thereof, in each case as the free base or in the form of a pharmaceutically acceptable salt.

In the pharmaceutical compositions according to the invention, the active ingredient content to be released in delayed manner is preferably between 0.5 and 85 wt. % and the content of pharmaceutically acceptable matrix former is between 8 and 40 wt. %. Particularly preferred pharmaceutical preparations are those with an active ingredient content to be released in delayed manner of between 3 and 70 wt. %, in particular of between 8 and 66 wt. %, and a content of pharmaceutically acceptable matrix former of between 10 and 35 wt. %, in particular of between 10 and 30 wt. %. If racemic (1RS,3RS,6RS)-6-dimethylaminomethyl-1-(3-methoxyphenyl)-cyclohexane-1,3-diol is used as the active ingredient, it is particularly preferred for the active ingredient content to be at the lower limit, i.e. between 0.5 and 25 wt. % (relative to total weight).

Further constituents of the matrix of the formulation according to the invention may be optionally digestible long-chain (i.e. with 8 to 50 C atoms, preferably 12 to 40 C atoms) unsubstituted or substituted hydrocarbons, such as for example fatty alcohols, fatty acid glyceryl esters, mineral and vegetable oils, as well as waxes, wherein hydrocarbons with a melting point of between 25° and 90° C. are preferred. In particular, fatty alcohols are preferred, most particularly lauryl alcohol, myristyl alcohol, stearyl alcohol, cetyl alcohol and cetyl stearyl alcohol. The content thereof in the matrix is 0 to 60 wt. %. Alternatively or additionally, the matrix may also have a content of polyethylene glycols of 0 to 60 wt. %.

The pharmaceutical formulations according to the invention may moreover contain pharmaceutically usual auxiliary substances as additional constituents, such as fillers, for example lactose, microcrystalline cellulose (MCC) or calcium hydrogenphosphate, as well as slip, lubricant and flow-control agents, for example talcum, magnesium stearate, stearic acid and/or highly disperse silicon dioxide, the total content of which in the tablets is between 0 and 80 wt. %, preferably between 5 and 65 wt. %.

The speed of release of an active ingredient from a dosage form is often dependent upon the pH value of the release medium. As the pharmaceutical preparation passes through the gastrointestinal tract, this pH value may vary within a range from below 1 to approximately 8. These variations may differ from one person taking the preparation to another. A different pH value/time profile on passage through the gastrointestinal tract may also be encountered in the same individual when the preparation is taken on different occasions. If the speed of release of the active ingredient from the pharmaceutical preparation is dependent upon the pH value, this may result in different speeds of release in vivo and thus differing bioavailability. Surprisingly, however, the release profiles of the active ingredient (in the form of the base or a pharmaceutically acceptable salt thereof) from a pharmaceutical formulation according to the invention are independent of the pH value, such as may occur physiologically on passage through the gastrointestinal tract. The release profiles at an ambient pH value of 1.2, 4.0 and 6.8 are identical both among themselves and in comparison with release over a pH value/time profile from pH 1.2 to above pH 2.3 and from pH 6.8 to pH 7.2.

It has been found that, for the purposes of achieving delayed active ingredient release from the formulation according to the invention, which is preferably in tablet form, it is immaterial, subject to otherwise unchanged dimensions and unchanged composition of the tablet with regard to the active ingredient, the matrix former and the optional constituents, whether the filler used is a water-soluble filler, for example lactose, an insoluble filler which does not swell in an aqueous medium, for example calcium hydrogenphosphate, or an insoluble filler which swells in an aqueous medium, for example microcrystalline cellulose. All such pharmaceutical preparations exhibit corresponding release behavior.

It is furthermore surprising that, in the compositions according to the invention, at a given quantity of active ingredient, the quantity of matrix former and the quantity of optional constituents may in each case vary over a relatively wide range without affecting the therapeutic effectiveness for at least 12 h (or 24 h) on twice (or once) daily administration (subject to compliance with the above-stated quantity limits for active ingredient, matrix former and further, optional constituents). Effectiveness over at least 12 h is ensured, for example, at an active ingredient content of approximately 32.25 wt. % (relative to the weight of the overall composition) not only in a composition comprising approximately 12.9 wt. % HPMC with a viscosity of 100,000 mPa·s as matrix former and a content of, for example MCC, as filler of approximately 52.6 wt. %, but also in a composition comprising approximately 25.8 wt. % of the same HPMC and approximately 39.7 wt. % of MCC (or lactose monohydrate) with otherwise identical quantities of slip, lubricant and flow-control agents. A comparable situation applies to compositions according to the invention having a higher or lower active ingredient content within the stated limits.

The formulation according to the invention contains the active ingredient 6-dimethylaminomethyl-1-(3-methoxyphenyl)-cyclohexane-1,3-diol as such and/or as a pharmaceutically acceptable salt in a quantity of conventionally to 1600 mg, in particular of 10 to 800 mg, very particularly preferably of 20 to 500 mg (weight of the active ingredient 6-dimethylaminomethyl-1-(3-methoxyphenyl)-cyclohexane-1,3-diol as the hydrochloride) per dosage unit, wherein the release behavior of the formulation according to the invention is not influenced by the exact quantity of the active ingredient, provided that the above-stated content limits are complied with.

Pharmaceutically acceptable salts of the active ingredient for the purposes of the present invention are such salts of the active ingredient, which, when used pharmaceutically, are physiologically compatible, in particular for use in mammals, especially humans. Such pharmaceutically acceptable salts may, for example, be formed with inorganic or organic acids. The hydrochloride salt is preferred.

The pharmaceutical compositions according to the invention may assume the form of both simple tablets and coated tablets, for example film tablets or sugar-coated tablets. The tablets are conventionally round and biconvex; oblong tablet shapes, which allow the tablet to be divided, are also possible. Granules, spheroids, pellets or microcapsules are also possible, which are packaged in sachets or capsules or may be compressed to form disintegrating tablets.

One or more coating layers may be used for the coated tablets. Known hydroxypropylmethylcelluloses with a low viscosity of approximately 1 to 100 mPa·s and a low molecular weight of <10,000 (for example Pharmacoat 606 with a viscosity of 6 mPa·s in a 2 wt. % aqueous solution at 20° C.), which have only a slight effect on the release profile of the pharmaceutical preparation according to the invention, are suitable as the coating material. Diffusion coatings known to the person skilled in the art, for example based on swellable, but water-insoluble poly(meth)acrylates, modulate the delay to active ingredient release from pharmaceutical formulations according to the invention. The tablet core, which contains the active ingredient and releases it in delayed manner, with an active ingredient content preferably of between 0.5 and 85 wt. %, particularly preferably of between 3 and 70 wt. % and very particularly preferably of between 8 and 66 wt. % may be covered with additional active ingredient, which is released in undelayed manner as an initial dose, by various methods known to the person skilled in the art, for example pan coating, spraying of solutions or suspensions or by powder application methods, without this being absolutely essential for the desired delayed release simultaneously accompanied by rapid loading of the active ingredient for rapid pain relief on first administration of the pharmaceutical formulation according to the invention. Multilayer and jacketed tablets are further embodiments in which 6-dimethylaminomethyl-1-(3-methoxyphenyl)-cyclohexane-1,3-diol or a pharmaceutically acceptable salt thereof is released in delayed manner by a pharmaceutically acceptable matrix former from one or more layers of the multilayer tablet with an active ingredient content preferably of between 0.5 and 85 wt. %, particularly preferably of between 3 and 70 wt. % and very particularly preferably of between 8 and 66 wt. % or from the core of the jacketed tablet with an active ingredient content preferably of between 0.5 and 85 wt. %, particularly preferably of between 3 and 70 wt. % and very particularly preferably of between 8 and 66 wt. %, while the active ingredient is released in undelayed manner from one or more layers of the multilayer tablet or from the outer jacket layer of the jacketed tablets. Multilayer and jacketed tablets may contain one or more coatings which contain no active ingredient.

Instead of a delayed release matrix in the pharmaceutical composition with delayed release, it is also possible to use a normal release matrix together with a coating which delays release of the active ingredient. The active ingredient may, for example, be present in a conventional matrix of microcrystalline cellulose and optionally further pharmaceutical auxiliary substances, such as for instance binders, fillers, slip, lubricant and flow-control agents, which are covered or coated with a material which controls delayed release of the active ingredient in an aqueous medium. Suitable coating materials are, for example, water-insoluble waxes and polymers, such as polymethacrylates (Eudragit or the like) or water-insoluble celluloses, in particular ethylcellulose. The coating material may optionally also contain water-soluble polymers, such as polyvinylpyrrolidone, water-soluble celluloses, such as hydroxypropylmethylcellulose or hydroxypropylcellulose, other water-soluble agents, such as Polysorbate 80, or hydrophilic pore formers, such as polyethylene glycol, lactose or mannitol.

In addition or as a complement to the possibilities of a controlled release matrix in the pharmaceutical formulation with delayed release or of a normal release matrix with a coating which delays release of the active ingredient, an osmotically driven release system may also be used to achieve delayed release. In such a, preferably oral, release system, at least one, preferably all, of the surfaces of the release system, preferably the layer(s) which is/are or could be in contact with the release medium, is/are semipermeable, preferably is/are provided with a semipermeable coating, such that the surface(s) is/are permeable to the release medium but is/are substantially, preferably completely, impermeable to the active ingredient, wherein the surface(s) and/or optionally the coating comprise(s) at least one opening for release of the active ingredient. In this case, the active ingredient 6-dimethylaminomethyl-1-(3-methoxyphenyl)-cyclohexane-1,3-diol or a pharmaceutically acceptable salt thereof, preferably (1RS,3RS,6RS)-6-dimethylaminomethyl-1-(3-methoxyphenyl)-cyclohexane-1,3-diol or a pharmaceutically acceptable salt thereof may, but need not, be present in a matrix. This should preferably be taken to mean a system in tablet form comprising a release opening, an osmotic pharmaceutical preparation core, a semipermeable membrane and a polymeric portion which exerts pressure. One good and preferred example of such a system is the OROS® System from the ALZA Corporation, USA, whose website or other product information contains details of the OROS® System. In particular, these systems are also the OROS® Push-Pull™ System, the OROS® Delayed Push-Pull™ System, the OROS® Multi-Layer Push-Pull™ System, the OROS® Push-Stick System, or also in certain cases, L-OROS®. Embodiments and examples of the actual production of osmotically driven release systems may be found in U.S. Pat. No. 4,765,989, U.S. Pat. No. 4,783,337 and U.S. Pat. No. 4,612,008, the complete content of which is incorporated herein by reference.

Another possibility for delayed release is a parenteral implant. This should be taken to mean any kind of non-biodegradable implant which slowly releases active ingredient over an extended period of time. One example is ALZA's DUROS SYSTEM, as is described for example in WO 00/54745 and consists of an inert tube, a semipermeable membrane, an “osmotic engine”, a plunger, a release opening and a reservoir to accommodate the (usually highly concentrated) active ingredient solution which is to be released. Suitable examples are described in patents U.S. Pat. No. 4,612,008, U.S. Pat. No. 4,765,989, U.S. Pat. No. 4,783,337, U.S. Pat. No. 5,264,446, U.S. Pat. No. 4,519,801, U.S. Pat. No. 4,612,008, U.S. Pat. No. 4,783,337 and U.S. Pat. No. 5,082,668. Another example is based on non-biodegradable polymer based on ethylene/vinyl acetate copolymer, such as is described, for example, for contraceptives by De Nijs et al. (U.S. Pat. No. 4,957,119, U.S. Pat. No. 5,088,505).

Another possibility for delayed release is a multipore tablet. Examples of this are the products developed by Gacell, Andrx, Elan (for example using MODAS, SODAS technology). Suitable examples may be found in EP 122077 A2, EP360562 B1, EP 320 097 A1 and U.S. Pat. No. 499,276.

Another possibility for delayed release is a gel/matrix tablet. Examples of this are the products developed by Penwest Pharmaceuticals (for example using TimeRX technology). Suitable examples may be found in U.S. Pat. No. 5,330,761, U.S. Pat. No. 5,399,362, U.S. Pat. No. 5,472,711 and U.S. Pat. No. 5,455,046.

Another possibility for delayed release is a transdermal administration system. These are taken to be systems which, optionally with the use of penetration auxiliaries such as softeners and penetration accelerators, are applied onto the skin and release the active ingredient into the body through the skin. Examples, all of which may also be used in the present case, are described inter alia in DE 10033853, U.S. Pat. No. 5,411,740, EP 767659, AT185694E and DE 69326848T2. Further examples directly transferable to the formulation are the suitable dressings from EP 0 430 019 B1, WO 98/36728 or WO 96/19975.

Another possibility for delayed release is a parenteral depot system, in particular depot systems based on polymers which slowly break down or biodegrade. Examples are polylactide polymers or polyglycolide polymers or in particular polylactide/polyglycolide copolymers (PLGA). Examples which are best known to the person skilled in manufacture are made by Alkermes or Medisorb and in particular for Enantone and Trenantone by Takeda. Such systems also include, however, injectable gels, in particular those which solidify in situ and slowly release the active ingredient dissolved therein. Examples are the Atrigel technology and other systems from Atrix (U.S. Pat. No. 5,278,201, U.S. Pat. No. 5,739,176, U.S. Pat. No. 6,143,314), in which PLGA polymers and active ingredients are mixed with pharmaceutically compatible solvents, which, once introduced into the body, solidify to form an implant, and the SABER technology from DURECT, which uses a three to four component system comprising sucrose acetate isobutyrate (SAIB), a pharmaceutically acceptable solvent, such as for example ethanol, and one or more additives and, of course, the active ingredient. Such systems also include ALZA's ALZAMER technology, in which stabilised particles in a thick PLGA polymer solution are injected. Another example may be found in EP729357.

All the products according to the invention are produced in a similar manner to the above-stated known products, such that, for the person skilled in the art, the production processes, structure and composition of these known products, which are straightforwardly available from product information or websites or patent applications/granted patents, are part of the disclosure of the present application. The contents of each of the above-stated granted patents or published patent applications are incorporated herein by reference in their entirety, as is the content of AU 1256399 and AU 9052298.

The present invention also provides a tablet for twice daily oral administration of 6-dimethylaminomethyl-1-(3-methoxyphenyl)-cyclohexane-1,3-diol containing a pharmaceutical formulation according to the invention.

The present invention also provides a tablet for once daily oral administration of 6-dimethylaminomethyl-1-(3-methoxyphenyl)-cyclohexane-1,3-diol containing a pharmaceutical formulation according to the invention.

The compositions according to the invention may, for example, be produced in accordance with the following general process: the constituents of the composition (active ingredient, matrix former and optional constituents) are weighed out in succession and then screened in a conventional screening machine. The Quadro Comil U10 screening machine may be used for this purpose, a normal screen size being approximately 0.813 mm. The screened composition is then mixed in a container mixer, for example in a Bohle container mixer; typical operating conditions are: duration approximately 15 min±45 s at a rotational speed of 20±1 rpm. The resultant powder mixture is then pressed in a tabletting press to form a tablet. A Korsch EKO tabletting press with a 10 mm diameter round, biconvex die may be used for this purpose. Alternatively, the powder mixture may also be compacted and the compression moldings subsequently screened (Comill 3 mm abrasive cutting screen followed by 1.2 mm round hole screen), the resultant granular product then being pressed as described above with the addition of lubricant (for example magnesium stearate), for example on an EKO tabletting press with 10 mm round dies. Granulation may also be performed by wet granulation using aqueous or organic solvents; aqueous solvents with or without suitable binders are preferred. The production process can straightforwardly be adapted to particular requirements and the desired dosage form in accordance with methods well known in the prior art.

The production of pharmaceutical compositions according to the invention is characterized by elevated reproducibility of the release characteristics of the compositions obtained, which contain 6-dimethylaminomethyl-1-(3-methoxyphenyl)-cyclohexane-1,3-diol or a pharmaceutically acceptable salt thereof. The release profile of pharmaceutical preparations according to the invention has proven to be stable over a period of storage of at least one year under conventional storage conditions in accordance with the ICH Q1AR stability testing guideline.

If taken once or twice daily, a pharmaceutical formulation according to the invention reliably achieves good therapeutic effectiveness in patients with chronic, severe pain.

### EXAMPLES

The Examples serve to illustrate the present invention and preferred embodiments, but are not intended to restrict its scope of protection.

### Example 1

Matrix tablets with the following composition per tablet

| | | |
|---|---|---|
| 6-Dimethylaminomethyl-1-(3-methoxyphenyl)-cyclohexane- | 203 | mg |
| 1,3-diol (with 1.5% water content) corresponding to 200 mg | | |
| of active ingredient | | |
| Hydroxypropylmethylcellulose (Metolose 90 SH 100,000 | 80 | mg |
| from Shinetsu), 100,000 mPa.s | | |
| Microcrystalline cellulose (Avicel PH 101 from FMC) | 61 | mg |
| Highly disperse silicon dioxide | 3 | mg |
| Magnesium stearate | 3 | mg |
| Total quantity | 350 | mg |

produced in the following manner in a batch size of 2000 tablets: All the constituents were weighed out and screened in a Quadro Comil U10 screening machine using a screen size of 0.813 mm, mixed in a container mixer (Bohle LM 40) for 15 min±15 s at a rotational speed of 20±1 rpm and pressed on a Korsch EKO eccentric press to form biconvex tablets with a diameter of 10 mm, a radius of curvature of 8 mm and an average tablet weight of 350 mg.

In vitro release was determined using the Ph. Eur. paddle method at 75 rpm in 900 ml of pH 6.8 buffer to Ph. Eur. at 37° C., with detection by UV spectrometry, and is shown in the following Table.

| | Total quantity of active |
|---|---|
| Time [min] | ingredient released [%] |
| 0 | 0 |
| 30 | 20 |
| 60 | 31 |
| 120 | 45 |
| 240 | 65 |
| 360 | 78 |
| 480 | 86 |
| 600 | 91 |
| 720 | 94 |
| 840 | 96 |
| 1080 | 98 |
| 1440 | 100 |

### Example 2

Matrix tablets with the following composition per tablet

| | | |
|---|---|---|
| 6-Dimethylaminomethyl-1-(3-methoxyphenyl)-cyclohexane- | 406 | mg |
| 1,3-diol (with 1.5% water content) corresponding to 400 mg | | |
| of active ingredient | | |
| Hydroxypropylmethylcellulose (Metolose 90 SH 100,000 | 160 | mg |
| from Shinetsu), 100,000 mPa.s | | |
| Microcrystalline cellulose (Avicel PH 101 from FMC) | 22 | mg |
| Highly disperse silicon dioxide | 6 | mg |
| Magnesium stearate | 6 | mg |
| Total quantity | 600 | mg |

were produced in the following manner in a batch size of 2000 tablets: All the constituents were weighed out and screened in a Quadro Comil U10 screening machine using a screen size of 0.813 mm, mixed in a container mixer (Bohle LM 40) for 15 min±15 s at a rotational speed of 20±1 rpm and pressed on a Korsch EKO eccentric press to form biconvex tablets with a diameter of 13 mm, a radius of curvature of 15 mm and an average tablet weight of 600 mg.

In vitro release was determined using the European Pharmacopoeia (“Ph. Eur.”) paddle method at 75 rpm in 900 ml of pH 6.8 buffer to Ph. Eur. at 37° C., with detection by WV spectrometry, and is shown in the following Table.

| | Total quantity of active |
|---|---|
| Time [min] | ingredient released [%] |
| 0 | 0 |
| 30 | 15 |
| 60 | 23 |
| 120 | 33 |
| 240 | 49 |
| 360 | 61 |
| 480 | 71 |
| 600 | 79 |
| 720 | 86 |
| 840 | 89 |
| 1080 | 97 |
| 1440 | 100 |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed:

1. A delayed-release pharmaceutical composition comprising as active ingredient 6-dimethylaminomethyl-1-(3-methoxyphenyl)-cyclohexane-1,3-diol or a pharmaceutically acceptable salt thereof in a delayed-release matrix,
wherein the matrix comprises 1 to 80 wt. % of at least pharmaceutically acceptable matrix forming polymer, and
wherein the pharmaceutical composition releases in vitro
3-35 wt. % of the active ingredient after 0.5 hours,
5-50 wt. % of the active ingredient after 1 hour,
10-75 wt. % of the active ingredient after 2 hours,
15-82 wt. % of the active ingredient after 3 hours,
30-97 wt. % of the active ingredient after 6 hours,
more than 50 wt. % of the active ingredient after 12 hours,
more than 70 wt. % of the active ingredient after 18 hours, and
more than 80 wt. % of the active ingredient after 24 hours,
as measured using a Ph. Eur. paddle method at 75 rpm in a buffer at a pH value of 6.8 at 37° C. and with detection by UV spectrometry.

2. A pharmaceutical composition according to claim 1, wherein the pharmaceutically acceptable matrix former comprises cellulose ethers, or cellulose esters, or both, which exhibit a viscosity in a 2 wt. % aqueous solution at 20° C. of from 10,000 to 150,000 mPa·s.

3. A pharmaceutical composition according to claim 2, wherein the pharmaceutically acceptable matrix former comprises cellulose ethers, or cellulose esters, or both, which exhibit a viscosity in a 2 wt. % aqueous solution at 20° C. of from 50,000 to 150,000 mPa·s.

4. A pharmaceutical composition according to claim 1, wherein the at least one pharmaceutically acceptable matrix forming polymer comprises at least one substance selected from the group consisting of hydroxypropylmethylcelluloses, hydroxyethylcelluloses, hydroxypropylcelluloses, methylcelluloses, ethylcelluloses and carboxymethylcelluloses.

5. A pharmaceutical composition according claim 4, wherein the at least one pharmaceutically acceptable matrix forming polymer comprises at least one substance selected from the group consisting of hydroxypropylmethylcelluloses, hydroxyethylcelluloses and hydroxypropylcelluloses.

6. A pharmaceutical composition according to claim 1, wherein the content of 6-dimethylaminomethyl-1-(3-methoxyphenyl)-cyclohexane-1,3-diol or salt thereof is between 0.5 and 85 wt. % and the content of the at least one pharmaceutically acceptable matrix forming polymer is between 8 and 40 wt. %.

7. A pharmaceutical composition according to claim 6, wherein the content of 6-dimethylaminomethyl-1-(3-methoxyphenyl)-cyclohexane-1,3-diol or salt thereof is between 3 and 70 wt. %, and the content of the at least one pharmaceutically acceptable matrix forming polymer is between 10 and 35 wt. %.

8. A pharmaceutical composition according to claim 7, wherein the content of he content of 6-dimethylaminomethyl-1-(3-methoxyphenyl)-cyclohexane-1,3-diol or salt thereof is between 8 and 66 wt. %, and the content of the at least one pharmaceutically acceptable matrix forming polymer is between 10 and 30 wt. %.

9. A pharmaceutical composition according to claim 1, comprising (1RS,3RS,6RS)-6-dimethylaminomethyl-1-(3-methoxyphenyl)-cyclohexane-1,3-diol or a pharmaceutically acceptable salt thereof.

10. A pharmaceutical composition according to claim 1, comprising 6-dimethylaminomethyl-1-(3-methoxyphenyl)-cyclohexane-1,3-diol as a racemic mixture, or as a mixture of stereoisomers thereof in any mixing ratio, in each case as the free base or in the form of a pharmaceutically acceptable salt.

11. A pharmaceutical composition according to claim 1, comprising a pure stereoisomer of 6-dimethylaminomethyl-1-(3-methoxyphenyl)-cyclohexane-1,3-diol, as the free base or in the form of a pharmaceutically acceptable salt.

12. An oral tablet comprising a pharmaceutical composition of claim 1 which provides effective administration of 6-dimethylaminomethyl-1-(3-methoxyphenyl)-cyclohexane-1,3-diol for 12 hours.

13. An oral tablet comprising a pharmaceutical composition of claim 1 which provides effective administration of 6-dimethylaminomethyl-1-(3-methoxyphenyl)-cyclohexane-1,3-diol for 24 hours.

14. A delayed-release pharmaceutical composition comprising as active ingredient 6-dimethylaminomethyl-1-(3-methoxyphenyl)-cyclohexane-1,3-diol or a pharmaceutically acceptable salt thereof in a delayed-release matrix,
wherein the matrix comprises 1 to 80 wt. % of at least one pharmaceutically acceptable matrix forming polymer, and
wherein the pharmaceutical composition releases in vitro
3-60 wt. % of the active ingredient after 0.5 hours,
5-70 wt. % of the active ingredient after 1 hour,
10-75 wt. % of the active ingredient after 2 hours,
15-82 wt. % of the active ingredient after 3 hours,
30-97 wt. % of the active ingredient after 6 hours,
more than 50 wt. % of the active ingredient after 12 hours,
more than 70 wt. % of the active ingredient after 18 hours, and
more than 80 wt. % of the active ingredient after 24 hours,
as measured using a Ph. Eur. paddle method at 75 rpm in a buffer at a pH value of 6.8 at 37° C. and with detection by UV spectrometry.

15. A pharmaceutical composition according to claim 14, wherein the at least one pharmaceutically acceptable matrix forming polymer comprises cellulose ethers, or cellulose esters, or both, which exhibit a viscosity in a 2 wt. % aqueous solution at 20° C. of from 10,000 to 150,000 mPa·s.

16. A pharmaceutical composition according to claim 15, wherein the at least one pharmaceutically acceptable matrix forming polymer comprises cellulose ethers, or cellulose esters, or both, which exhibit a viscosity in a 2 wt. % aqueous solution at 20° C. of from 50,000 to 150,000 mPa·s.

17. A pharmaceutical composition according to claim 14, wherein the at least one pharmaceutically acceptable matrix forming polymer comprises at least one substance selected from the group consisting of hydroxypropylmethylcelluloses, hydroxyethylcelluloses, hydroxypropylcelluloses, methylcelluloses, ethylcelluloses and carboxymethylcelluloses.

18. A pharmaceutical composition according claim 17, wherein the at least one pharmaceutically acceptable matrix forming polymer comprises at least one substance selected from the group consisting of hydroxypropylmethylcelluloses, hydroxyethylcelluloses and hydroxypropylcelluloses.

19. A pharmaceutical composition according to claim 14, wherein the content of 6-dimethylaminomethyl diol or salt thereof is between 0.5 and 85 wt. % and the content of pharmaceutically acceptable matrix former is between 8 and 40 wt. %.

20. A pharmaceutical composition according to claim 19, wherein the content of 6-dimethylaminomethyl is between 3 and 70 wt. %, and the content of the at least one pharmaceutically acceptable matrix forming polymer is between 10 and 35 wt. %.

21. A pharmaceutical composition according to claim 20, wherein the content of 6-dimethylaminomethyl is between 8 and 66 wt. %, and the content of the at least one pharmaceutically acceptable matrix forming polymer is between 10 and 30 wt. %.

22. A pharmaceutical composition according to claim 14, comprising (1RS,3RS,6RS)-6-dimethylaminomethyl-1-(3-methoxyphenyl)-cyclohexane-1,3-diol or a pharmaceutically acceptable salt thereof.

23. A pharmaceutical composition according to claim 14, comprising 6-dimethylaminomethyl-1-(3-methoxyphenyl)-cyclohexane-1,3-diol as a racemic mixture, or as a mixture of stereoisomers thereof in any mixing ratio, or as a pure stereoisomer thereof, in each case as the free base or in the form of a pharmaceutically acceptable salt.

24. A pharmaceutical composition according to claim 14, comprising a pure stereoisomer of 6-dimethylaminomethyl-1-(3-methoxyphenyl)-cyclohexane-1,3-diol, as the free base or in the form of a pharmaceutically acceptable salt.

25. An oral tablet comprising a pharmaceutical composition of claim 14 which provides effective administration of 6-dimethylaminomethyl-1-(3-methoxyphenyl)-cyclohexane-1,3-diol for 12 hours.

26. An oral tablet comprising a pharmaceutical composition of claim 14 which provides effective administration of 6-dimethylaminomethyl-1-(3-methoxyphenyl)-cy-clohexane-1,3-diol for 24 hours.

27. A delayed-release pharmaceutical composition comprising as active ingredient 6-dimethylaminomethyl-1-(3-methoxyphenyl)-cyclohexane-1,3-diol or a pharmaceutically acceptable salt thereof in a delayed-release matrix, wherein the matrix comprises 1 to 80 wt. % of at least one pharmaceutically acceptable matrix forming polymer, wherein the at least one pharmaceutically acceptable matrix forming polymer comprises cellulose ethers, or cellulose esters, or both, which exhibit a viscosity in a 2 wt. % aqueous solution at 20° C. of from 3,000 to 150,000 mPa·s.

28. A pharmaceutical composition according to claim 27, wherein the at least one pharmaceutically acceptable matrix forming polymer comprises cellulose ethers, or cellulose esters, or both, which exhibit a viscosity in a 2 wt. % aqueous solution at 20° C. of from 10,000 to 150,000 mPa·s.

29. A pharmaceutical composition according to claim 28, wherein the at least one pharmaceutically acceptable matrix forming polymer comprises cellulose ethers, or cellulose esters, or both, which exhibit a viscosity in a 2 wt. % aqueous solution at 20° C. of from 50,000 to 150,000 mPa·s.

30. A pharmaceutical composition according to claim 29, wherein the at least one pharmaceutically acceptable matrix forming polymer comprises at least one substance selected from the group consisting of hydroxypropylmethylcelluloses, hydroxyethylcelluloses, hydroxypropylcelluloses, methylcelluloses, ethylcelluloses and carboxymethylcelluloses.

31. A pharmaceutical composition according claim 30, wherein the at least one pharmaceutically acceptable matrix forming polymer comprises at least one substance selected from the group consisting of hydroxypropylmethylcelluloses, hydroxyethylcelluloses and hydroxypropylcelluloses.

32. A pharmaceutical composition according to claim 27, wherein the content of 6-dimethylaminomethyl-1-(3-methoxyphenyl)-cyclohexane-1,3-diol or salt thereof is between 0.5 and 85 wt. % and the content of the at least one pharmaceutically acceptable matrix forming polymer is between 8 and 40 wt. %.

33. A pharmaceutical composition according to claim 32, wherein the content of 6-dimethylaminomethyl-1-(3-methoxyphenyl)-cyclohexane-1,3-diol is between 3 and 70 wt. %, and the content of pharmaceutically acceptable matrix forming polymer is between 10 and 35 wt. %.

34. A pharmaceutical composition according to claim 33, wherein the content of 6-dimethylaminomethyl diol is between 8 and 66 wt. %, and the content of pharmaceutically acceptable matrix forming polymer is between 10 and 30 wt. %.

35. A pharmaceutical composition according to claim 27, comprising (1RS,3RS,6RS)-6-dimethylaminomethyl-1-(3-methoxyphenyl)-cyclohexane-1,3-diol or a pharmaceutically acceptable salt thereof.

36. A pharmaceutical composition according to claim 27, comprising 6-dimethylaminomethyl-1-(3-methoxyphenyl)-cyclohexane-1,3-diol as a racemic mixture, or as a mixture of stereoisomers thereof in any desired mixing ratio, or as a pure stereoisomer thereof, in each case as the free base or in the form of a pharmaceutically acceptable salt.

37. A pharmaceutical composition according to claim 27, comprising a pure stereoisomer of 6-dimethylaminomethyl-1-(3-methoxyphenyl)-cyclohexane-1,3-diol, as the free base or in the form of a pharmaceutically acceptable salt.

38. An oral tablet comprising a pharmaceutical composition of claim 27 which provides effective administration of 6-dimethylaminomethyl-1-(3-methoxyphenyl)-cyclohexane-1,3-diol for 12 hours.

39. An oral tablet comprising a pharmaceutical composition of claim 27 which provides effective administration of 6-dimethylaminomethyl-1-(3-methoxyphenyl)-cyclohexane-1,3-diol for 24 hours.
